(19) United States
(12) Patent Application Publication
(10) Pub. No.: US 2023/0174452 A1
(43) Pub. Date: Jun. 8, 2023

(54) METHOD FOR BREAKING DOWN MICHAEL ADDUCTS CONTAINED IN A FLUID F AND FORMED DURING THE PREPARATION OF ACRYLIC ACID

(71) Applicant: BASF SE, Ludwigshafen am Rhein (DE)

(72) Inventors: Tile GIESHOFF, Ludwigshafen am Rhein (DE); Ulrich HAMMON, Ludwigshafen am Rhein (DE); Christian RAITH, Ludwigshafen am Rhein (DE); Volker SCHLIEPHAKE, Ludwigshafen am Rhein (DE); Anna Katharina DUERR, Shanghai (CN); Tim VAN LOOVEREN, Antwerp (BE); Christian REIN, Ludwigshafen am Rhein (DE)

(21) Appl. No.: 17/921,997

(22) PCT Filed: Apr. 26, 2021
(86) PCT No.: PCT/EP2021/060803

§ 371 Date: Oct. 28, 2022

(30) Foreign Application Priority Data
May 4, 2020 (EP) 20172654.4

(51) Int. Cl. C07C 51/44

(52) U.S. Cl. CPC C07C 51/44

(57) ABSTRACT

The present invention relates to a method of redissociating Michael adducts of acrylic acid present in a liquid F in a redissociation apparatus comprising at least one separating column K, an evaporator V and a pump P, wherein, in the event of an unwanted rise in the viscosity of the residue R in the bottom space of the separating column K, the feed of the liquid F into the redissociation apparatus is stopped, the residue R in the bottom space of the separating column K is diluted and cooled with a solvent 1, and the bottom space of the separating column K is emptied.

### DESCRIPTION

The present invention relates to a method of redissociating Michael adducts of acrylic acid present in a liquid F in a redissociation apparatus comprising at least one separating column K, an evaporator V and a pump P, wherein, in the event of an unwanted rise in the viscosity of the residue R in the bottom space of the separating column K, the feed of the liquid F into the redissociation apparatus is stopped, the residue R in the bottom space of the separating column K is diluted and cooled with a solvent 1, and the bottom space of the separating column K is emptied.

Acrylic acid is an important intermediate which finds use, for example, in the preparation of polymer dispersions (including in the form of their esters with alcohols) and superabsorbents.

Acrylic acid is obtainable, inter alia, by heterogeneously catalyzed gas phase partial oxidation of C₃ precursor compounds of acrylic acid (this term is intended to encompass, more particularly, those chemical compounds which are obtainable in a formal sense by reduction of acrylic acid; known C₃ precursors of acrylic acid are, for example propane, propene, acrolein, propionaldehyde and propionic acid; however, the term is also intended to encompass precursor compounds of the aforementioned compounds, for example glycerol (proceeding from glycerol, acrylic acid can be obtained, for example, by heterogeneously catalyzed oxidative dehydration in the gas phase; cf., for example, EP 1 710 227 A, WO 2006/114506 and WO 2006/092272)) with molecular oxygen over solid-state catalysts at elevated temperature (for example, DE 10 2007 055 086 A and DE 10 2006 062 258 A).

Owing to numerous parallel and subsequent reactions which proceed in the course of the catalytic gas phase partial oxidation and owing to the inert diluent gases which also have to be used in the course of the partial oxidation, what is obtained in the catalytic gas phase partial oxidation is not pure acrylic acid, but rather a reaction gas mixture (a product gas mixture) which comprises essentially acrylic acid, the inert diluent gases and by-products, and from which the acrylic acid has to be removed.

Typically, one way of removing the acrylic acid from the reaction gas mixture is to first convert the acrylic acid from the gas phase to the condensed (liquid) phase by employing absorptive and/or condensative measures. The further removal of the acrylic acid from the liquid phase thus obtained is subsequently typically undertaken by means of extractive, distillative, desorptive, crystallizative and/or other thermal separation processes.

Such an unwanted side reaction is free-radical polymerization with itself to form acrylic acid polymer or oligomer. What is disadvantageous about this side reaction is that it is essentially irreversible, which means that monomeric acrylic acid converted to free-radical acrylic acid polymer is lost to the acrylic acid production process and reduces the acrylic acid yield of the production process. What is advantageous about unwanted free-radical polymerization of acrylic acid is, however, that it can be at least reduced by addition of polymerization inhibitors.

This is not true of the second unwanted side reaction of acrylic acid in the liquid phase. This side reaction is what is called the Michael addition of an acrylic acid molecule onto another acrylic acid molecule to form a dimeric Michael adduct (“dimeric acrylic acid”), which can continue through further Michael addition of acrylic acid molecules (“monomeric acrylic acid”) onto already formed Michael adducts to form oligomeric Michael adducts (“oligomeric acrylic acid”).

Dimeric Michael adducts and oligomeric Michael adducts are to be referred to collectively in this document by the term “Michael adducts”. In the absence of the prefix “Michael” in this document, the terms “oligomer” and “polymer” mean the compounds that have resulted from free-radical reaction.

By contrast with free-radical polymerization of acrylic acid, the reactions for formation of the Michael adducts are typically reversible formation reactions. Since the boiling points of acrylic acid are below those of the Michael adducts (from which it has been reformed), the reformed acrylic acid can be removed continuously from the reaction equilibrium by superimposition of an appropriate pressure gradient, and hence the reverse reaction can be gradually completed.

A recovery of the acrylic acid chemically bound in the Michael adducts that has been brought about in this way is desirable in that this can increase the target product yield in the preparation of acrylic acid.

On account of comparatively high boiling points, the Michael adducts are generally obtained as a constituent of bottoms liquids in the thermal separation of liquid reaction product mixtures in the course of preparation of acrylic acid. Typically, such bottoms liquids, based on their weight, comprise 10% by weight of Michael adducts.

In addition, such liquids comprising Michael adducts, as well as acrylic acid, typically also comprise other constituents having boiling points different than those of the Michael adducts.

These boiling points may be either above or below those of the Michael adducts. Therefore, if liquids comprising Michael adducts that have been formed in the preparation of acrylic acid are subjected to a method of redissociation of the Michael adducts present therein by supply of thermal energy, the resulting splitting gas comprising at least a portion of the redissociation products is preferably subjected to a countercurrent rectification in order to recover the redissociation products present in the splitting gas with elevated purity (cf., for example, WO 2004/035514).

It is therefore customary to conduct a method of redissociating Michael adducts present in a liquid with a proportion by weight, based on the weight of the liquid, of 10% by weight, these having been formed in the preparation of acrylic acid, in a redissociation apparatus (cf., for example, WO 2010/066601).

However, it has been found to be disadvantageous in the redissociation method that the viscosity of the residue can rise unexpectedly during continuous operation.

EP 3 255 030 A teaches the addition of higher alcohols during the residue splitting, wherein maleic anhydride present in the residue is to be converted to less polymerization-sensitive maleic esters.

U.S. Pat. No. 6,414,183 teaches the dilution of the residue discharged with solvents such as acetic acid, water and methanol.

WO 2007/147651 describes the distillation of polymerization-sensitive residues in the presence of a boiling oil.

It is therefore an object of the present invention to provide an improved redissociation method.

Accordingly is a method of redissociating Michael adducts of acrylic acid that are present in a liquid F and have been formed in the preparation of acrylic acid, in which the liquid F comprises at least 10% by weight of Michael adducts of acrylic acid, based on the liquid F, in a redissociation apparatus comprising at least one separating column K which consists, from the bottom upward, of a bottom space, a separating space that comprises separating internals and adjoins the bottom space, and a top space adjoining the latter, and in which the pressure in the gas phase decreases from the bottom upward, an evaporator V and a pump P, in which Michael adducts present in the liquid F are split at a temperature of 130° C. to 240° C. and removed by distillation, and the remaining residue R is discharged, which comprises, in the event of an unwanted rise in the viscosity of the residue R in the bottom space of the separating column K, stopping the feed of the liquid F into the redissociation apparatus, diluting and cooling the residue R in the bottom space of the separating column K with at least 10% by volume of a solvent 1, based on the total volume of the residue R in the bottom space of the separating column K, and emptying the bottom space of the separating column K, where the solvent 1 has a boiling point at 1013 hPa of at least 150° C. and a solubility in water at 25° C. of at least 10 g per 100 g of water.

The liquid F comprises preferably at least 20% by weight of Michael adducts of acrylic acid, more preferably at least 30% by weight of Michael adducts of acrylic acid, most preferably at least 40% by weight of Michael adducts of acrylic acid, based in each case on the liquid F.

The Michael adducts present in the liquid F are split at a temperature of preferably 140 to 220° C., more preferably of 150 to 200° C., most preferably of 155 to 180° C.

The evaporation of the acrylic acid released in the redissociation can be assisted in various ways. The redissociation can, for example, be conducted under reduced pressure. It is alternatively possible that a stripping gas is guided into the splitting apparatus above the bottoms liquid and below the lowermost separating internals of the separating column K. In the latter case, it is advantageous, in the event of an unwanted rise in the viscosity of the residue in the bottom space of the separating column K, to stop the feed of stripping gas into the splitting apparatus.

The solvent 1 has a boiling point at 1013 hPa of preferably at least 170° C., more preferably at least 190° C., most preferably at least 210° C., and a solubility in water at 25° C. of preferably at least 20 g per 100 g of water, more preferably at least 30 g per 100 g of water, most preferably at least 40 g per 100 g of water.

The boiling point of solvent 1 at 1013 hPa should if at all possible be higher than the temperature of the residue R.

Suitable solvents 1 are, for example, alcohols such as ethylene glycol, propylene glycol, diethylene glycol, triethylene glycol and 2-ethoxyethanol, carboxamides such as N,N-dimethylacetamide, N-methylacetamide and N,N-dimethylformamide, sulfoxides such as dimethyl sulfoxide, and sulfones such as sulfolane. Rather than the pure substances, it is also possible to use residues comprising these substances. Suitable solvents 1 are, for example, residues from the oxo process.

The residue R in the bottom space of the separating column K, in the event of an unwanted rise in viscosity, is diluted with preferably at least 20% by volume, more preferably at least 30% by volume, most preferably at least 40% by volume, of solvent 1, based in each case on the total volume of the residue R in the bottom space of the separating column K.

The viscosity of the residue R in the bottom space of the separating column K at a temperature of 100° C. is preferably less than 12 Pa s, more preferably less than 10 Pa s, most preferably less than 8 Pa s.

The discharged residue R may be diluted with a solvent 2. Suitable solvents are, for example, alcohols, carboxamides, sulfoxides and sulfones. Rather than the pure substances, it is also possible to use residues comprising these substances. Suitable solvents are, for example, residues from methanol production.

Liquids F which comprise Michael adducts and have been formed in the preparation of acrylic acid are obtained, for example, in processes for preparing acrylic acid in which an acrylic acid-comprising product gas mixture obtained by catalytic gas phase partial oxidation of a C₃ precursor compound of acrylic acid, optionally after cooling, is fractionally condensed, ascending into itself, with side draw removal of a crude acrylic acid in a separating column provided with separating internals, and the liquid formed that comprises Michael adducts of acrylic acid is discharged continuously from the bottom of the condensation column and fed as liquid F to the redissociation of the Michael adducts of acrylic acid that are present therein (cf., for example, WO 2004/035514).

It will be appreciated that liquids F may also be the result when the acrylic acid present in the product gas mixture from the heterogeneously catalyzed gas phase partial oxidation is converted to the liquid phase by absorption into an absorbent and the acrylic acid is subsequently separated from the absorbate by means of rectificative and/or crystallizative separation methods, as disclosed by DE 103 36 386 A and DE 29 01 783 A.

In general, liquids F comprise, based on their weight, at least 10 ppm by weight, frequently at least 50 ppm by weight and in many cases at least 150 ppm by weight of polymerization inhibitor. In general, the content of polymerization inhibitors in liquids F on the same basis is not more than 1% by weight, or not more than 0.5% by weight. As well as phenothiazine (PTZ) and/or hydroquinone monomethyl ether (MEHQ) and conversion products thereof, useful polymerization inhibitors of this kind may also be compounds such as alkylphenols (e.g. o-, m- or p-cresol (methylphenol)), hydroxyphenols (e.g. hydroquinone), tocopherols (e.g. o-tocopherol) and N-oxyls (e.g. hydroxy-2,2,6,6-tetramethylpiperidine N-oxyl) and the other polymerization inhibitors known in the literature.

The constituents of liquids F other than acrylic acid and the Michael adducts are primarily compounds having a higher boiling point than acrylic acid at standard pressure.

Separating columns K used may in principle be any of the types of rectification column known per se.

These are all columns comprising separating internals, useful separating internals being, for example, structured packings, random packings and/or trays. The purpose of the separating internals is to increase the exchange area between gas phase ascending in the separating column K and liquid descending in the separating column K, and hence to improve mass transfer and heat transfer between the two phases. They are permeable both to gas ascending in the separating column K and to liquid descending in the separating column K.

The separating column K preferably comprises solely trays and/or structured packings. Trays used are advantageously dual-flow trays, and the separating column K particularly advantageously comprises exclusively dual-flow trays as separating internals.

Dual-flow trays are understood in this document to mean plates having simple passages (holes, slots etc.). The gas ascending in the separating column K and the liquid descending in the separating column flow counter to one another through the same passages. The cross section of the passages is matched in a known manner to the load on the separating column K. If it is too small, the ascending splitting gas will flow through the passages at such high speed that the liquid descending in the separating column K is entrained essentially without separating action. If the cross section of the passages is too high, ascending splitting gas and descending liquid will essentially move past one another without exchange, and there is a risk that the tray will run dry. Typically, dual-flow trays do not have a downcomer that connects them to the next tray. Each dual-flow tray may of course conclude flush with the walls of the rectification column. It may alternatively be connected thereto via lands. With decreasing load on the rectification column, dual-flow trays run dry, by contrast with hydraulically sealed crossflow trays.

The feed point I at which the liquid F is introduced into the separating column K is above the lowermost separating internals in the separating column K. In the case of a tray column, the feed point I is thus above the lowermost tray.

If the separating column K is a column with purely structured packing, the feed point I is above the lowermost structured packing.

A separating column K comprising purely dual-flow trays may comprise up to 60 dual-flow trays or more. Advantageously, these have an opening ratio (the ratio D:U, formed from the proportion of the tray area which is permeable to the splitting gas (D) and the total area of the tray (U)) of 10% to 20%, preferably of 10% to 15%.

Advantageously, the feed point I in the case of a tray column having purely dual-flow trays (for example with at most 40 equidistant dual-flow trays) is in the region of the fourth to tenth dual-flow tray viewed from the bottom upward. Appropriately in application terms, the feed temperature of the liquid F at the feed point I corresponds to the temperature of the liquid descending in the separating column K at that point. Advantageously, the two aforementioned temperatures differ from one another by not more than 10% (based on their arithmetic average). Appropriately in application terms, the separating column K and its feed and drain conduits are thermally insulated from the environment.

In general, separating columns K having 2 to 25 theoretical plates are sufficient. A theoretical plate is understood to mean that spatial unit of the separating space comprising separating internals in the separating column K that brings about enrichment of matter in accordance with the thermodynamic equilibrium without loss of energy.

Preferably, the feed point I of the separating column K is in the region of the second to eighth theoretical plate, viewed from the bottom upward.

The reflux liquid for the separating column K can be generated by direct and/or indirect cooling of the gas stream G flowing (in)to the top space of the separating column K. Advantageously in accordance with the invention, the method of direct cooling is employed.

For this purpose, in the simplest manner, the gas stream G flowing through the uppermost separating internals of the separating column K into the top space above is fed to a quench apparatus that may be integrated, for example, into the top space (in this case, the top space is separated from the separating space, for example by means of a chimney tray; bottom space and top space do not comprise any separating internals).

In principle, the quench apparatus may alternatively be disposed outside the separating column K. A useful quench apparatus of this kind may be any of the apparatuses known for this purpose in the prior art (for example spray scrubbers, Venturi scrubbers, bubble columns or other apparatuses with surfaces over which liquid trickles), preference being given to using Venturi scrubbers or spray coolers. Advantageously, a cocurrent apparatus (for example one with an impingement plate nozzle) is used. For indirect cooling of the quench liquid, it is typically guided through an (indirect) heat transferer or heat exchanger. All standard heat transferers or heat exchangers are suitable in this regard. Preference is given to shell and tube heat exchangers, plate heat exchangers and air coolers. Suitable cooling media are air in the case of the corresponding air cooler, and cooling liquids, especially water (e.g. surface water) in the case of the other cooling apparatus. Appropriately for application purposes, the quench liquid used is a portion of the condensate formed in the quenching operation. The other portion of the condensate formed in the quenching operation is normally recycled essentially as reflux liquid to the uppermost separating internals in the separating column K (if required, it is also possible to discharge a portion of the condensate). It is of course also possible to conduct the condensation exclusively with indirect heat exchangers integrated into the top space and/or outside the top space by passing the gas stream G through these.

Advantageously for application purposes, the separating column K is operated with inhibition of polymerization. Such polymerization inhibitors that may be used for this purpose are in principle any of the polymerization inhibitors known in the prior art for acrylic monomers. Examples of these include phenothiazine (PTZ) and hydroquinone monomethyl ether (MEHQ).

These two are frequently employed in combination. Appropriately, they are added to the refluxed liquid dissolved in pure redissociation product. MEHQ is preferably metered in in molten form.

Evaporators V used may in principle be any of the types of evaporator that are known per se.

For this purpose, in the simplest manner, residue R withdrawn as substream I in the bottom space of the separating column K is heated by means of an indirect circulation heat exchanger, and the residue R thus heated is recycled into the separating column as substream II via feed point II. The residue R is conveyed here by means of a pump P through the indirect circulation heat exchanger (forced circulation heat exchanger).

In the case of an indirect circulation heat exchanger, the heat is not transferred by direct contact between fluid heat carrier and liquid mixture to be heated which is forced by mixing. Instead, the heat is transferred indirectly between fluids separated by a dividing wall. The active separating area of the heat transferer (heat exchanger) which is active for the heat transfer is referred to as the heat exchange or transfer area, and the heat transfer follows the known laws of heat transfer.

Both the fluid heat carrier and residue R flow through the indirect circulation heat exchanger. In other words, both of these flow into the heat exchanger and back out again (one flows through the at least one primary space, and the other through the at least one secondary space).

Useful fluid heat carriers for the process according to the invention are in principle all possible hot gases, vapors and liquids.

The primary fluid heat carrier is steam, which may be at different pressures and temperatures. Frequently, it is favorable when the steam condenses as it passes through the indirect heat exchanger (saturated steam).

Indirect circulation heat exchangers suitable for the process are especially concentric tube, shell and tube, fin tube, spiral or plate heat transferers. Concentric tube heat transferers consist of two concentric tubes.

A plurality of these concentric tubes can be joined to form tube walls. The inner tube may be smooth or be provided with ribs to improve the heat transfer. In individual cases, a tube bundle can also replace the inner tube. The fluids exchanging heat may move in cocurrent or in countercurrent. Appropriately in accordance with the invention, the liquid F is conveyed upward in the inner tube and hot steam flows downward, for example in the ring space.

Shell and tube heat transferers are particularly suitable for the process according to the invention. They normally consist of a closed wide outer tube which encloses the numerous smooth or finned transferer tubes of small diameter, which are secured to the tube plates.

The feed point II (this is understood to mean the point in the bottom space of the separating column K at which substream II exits from the feed conduit into the bottom space) is below the lowermost separating internals of the separating column K and above the level S of the bottoms liquid (of the liquid effluxing into the bottom space of the separating column K). Advantageously in accordance with the invention, the level S of the liquid (the bottoms liquid) effluxing into the bottom spaces is adjusted such that it is less than 40%, preferably less than 30% and more preferably less than 20% of the distance A. In general, however, the level S will not be less than 5% of the distance A (safety liquid level).

Advantageously in accordance with the invention, this safety height is achieved with a small bottoms liquid volume in that displacement bodies are mounted in the bottom space or the bottom space is tapered towards its lower end (cf. FIG. 6 of DE 103 32 758 A or else EP 1 095 685 A and FIG. 1 of DE 10 2004 015 727 A).

Particularly advantageously, the bottom space is tapered towards its lower end, and the level S of the liquid effluxing into the bottom space (level of the bottoms liquid) is in the section of the bottom space in which the bottom space is tapered (i.e. in the section in which it has a reduced internal diameter).

In general, the feed point II is at least 0.25 x A above the level S of the bottoms liquid (above the liquid level of the bottoms liquid).

The substream II is recycled into the bottom space of the separating column K in such a way that the substream II is not directed onto the bottoms liquid in the bottom space of the separating column K (meaning that the extension of the flow vector of that flow with which substream II exits from the corresponding feed into the bottom space does not hit the bottoms liquid, but hits a solid article other than the bottoms liquid (i.e. the wall of the bottom space, a baffle plate etc.)).

In a simple manner, the aforementioned condition of the invention can be implemented in that the substream II flows horizontally into the bottom space (for example via a simple feed port).

Advantageously, however, substream II is fed into the bottom space of the separating column K from a conduit A conducted into the bottom space, the exit opening of which points downward in the bottom space, but is directed not onto the bottoms liquid but onto an impingement device A (directed onto a flow distributor) which is mounted in the bottom space above the level S of the bottoms liquid and which deflects substream II in an upward direction when it hits the impingement device (cf., for example, FIG. 1 of DE 10 2004 015 727 A).

Frequently, the forced circulation heat exchanger is also designed as a forced circulation flash heat transferer, preferably a forced circulation shell-and-tube flash heat transferer. By contrast with the case of a pure forced circulation heat transferer, this is normally separated from the feed point II in the separating column K by a throttle device (for example in the simplest case by a perforated plate (or other restrictor); a valve is an alternative option).

The above measure suppresses any boiling of the at least one substream I pumped in circulation within the at least one secondary space of the heat transferer (heat exchanger), for example in the tubes of the shell-and-tube heat transferer. The at least one substream I pumped in circulation is instead superheated within the at least one secondary space with regard to the gas pressure GD that exists in the bottom space of the separating column K, and the boiling process is thus moved completely to the passage side of the throttle device (meaning that the contents of the tubes of the shell-and-tube heat transferer are in monophasic form; the shell-and-tube heat transferer functions merely as a superheater). The throttle device separates the heat transferer (heat exchanger; e.g. shell-and-tube heat exchanger) and the feed point II on the pressure side and, through suitable choice of the output of the pump according to the invention, enables the establishment of a throttle supply pressure above the gas pressure GD that exists in the bottom space, which is above the gas pressure GD that exists in the bottom space, which is above the boiling pressure corresponding to the temperature of the substream II flowing out of the at least one secondary space of the heat transferer. The evaporative boiling takes place only beyond the throttle in flow direction. The use of forced circulation flash heat exchangers is preferred.

The difference between the throttle supply pressure and the gas pressure GD that exists in the bottom space here is typically 0.1 to 5 bar, frequently 0.2 to 4 bar and in many cases 1 to 3 bar.

In principle, the evaporator V may also be a thin-film evaporator integrated into the separating column K. The thin-film evaporator is between the separating space and the bottom space of the separating column K. Residue R withdrawn as substream in the bottom space of the separating column K is returned here to the thin-film evaporator by means of a pump P.

If “stripping gas” is used in part as entraining agent (entraining gas or else support gas) for the redissociation products (splitting products) in the separating column K, in the process of the invention, this is likewise guided into the bottom space of the separating column K above the level S of the bottoms liquid and below the lowermost separating internals of the separating column K (whence it flows into the top space of the separating column K). The latter is again done in such a way that the gas stream in the bottom space of the separating column K is not directed onto the bottoms liquid (i.e. the extension of the flow reactor with which the gas stream exits from the corresponding conduit into the bottom space does not hit the bottoms liquid).

This can be accomplished in a simple manner in that the stripping gas stream flows horizontally into the bottom space (for example via a simple feed port).

Advantageously, however, a stripping gas stream is fed into the bottom space of the separating column K from a conduit B conducted into the bottom space, the exit opening of which points downward in the bottom space, but is directed not onto the bottoms liquid but onto an impingement device B (directed onto a flow distributor) which is mounted in the bottom space above the level S of the bottoms liquid and which deflects the stripping gas stream in an upward direction when it hits the impingement device (cf., for example, FIG. 1 of DE 10 2004 015 727 A).

For reasons of inhibition of polymerization, the stripping gas preferably comprises molecular oxygen. Useful examples thereof include air, oxygen-depleted air and/or cycle gas. Cycle gas is understood here to mean the tail gas that remains when the acrylic acid is converted to the liquid state of matter by absorption with a liquid absorbent or by fractional condensation from the product gas mixture of the heterogeneously catalyzed gas phase partial oxidation of a C₃ pre-cursor compound (e.g. propene, propane, acrolein, glycerol) employed for preparation of acrylic acid (cf., for example, WO 2004/035514). The predominant amount of this tail gas is recycled into the partial oxidation in circulation in order to dilute the reaction gas mixture.

In general, an aqueous phase is condensed out of the aforementioned tail gas prior to use thereof as stripping gas, which generally comprises residual amounts of acrylic acid (acid water) that can be separated from this aqueous phase by extraction with an organic extractant into the resulting extract. Prior to any use of the tail gas as stripping gas in the process according to the invention, the tail gas may also have been used to strip the aforementioned extract free of acrylic acid (cf. DE 10 2007 004 960 A). Normally, the stripping gas is supplied at a temperature below the temperature of the bottoms liquid and above 100° C., in some cases above 150° C.

Based on 1 kg of liquid F supplied at feed point I per hour, the stripping gas stream supplied may, for example, be 1 to 100 kg/h. Stripping gas is used in part especially when the evaporator V is a forced circulation flash heat exchanger.

The metered addition of stripping gas allows the partial pressure of the (re)dissociation products in the separating column K to be lowered in a corresponding manner, in the same way as by the imposition (application) of reduced pressure.

If no stripping gas is fed into the separating column K, a working pressure which is advantageously below 1 bar (and is, for example, 100 mbar) is employed at the top of the column.

If a stripping gas is used in part, the working pressure at the top of the separating column K is generally at a pressure of 1 to 3 bar, preferably 1.5 to 2.5 bar.

The temperature of the bottoms liquid present in the bottom space with level S is generally in the range from 140 to 220° C., frequently 150 to 200° C. and in many cases 155 to 180° C.

A substream of the residue R is discharged as residual stream and sent to disposal, for example incineration.

The gas stream that remains in the partial condensation of the gas stream G and is discharged in the process of the invention may likewise be used further in the same way as any portion of the condensate formed which is not used as reflux liquid, as already described in the prior art (e.g. DE 103 32 758 A, WO 2004/035514, WO 2008/090190, WO 2008/077767, EP 0 780 360 A, DE 197 01 737 A and EP 1 357 105 A).

It will be appreciated that dispersants (e.g. surfactants) and/or defoamers may be added to the bottoms liquid of the separating column K, as recommended, for example, in DE 10 2008 001 435 A. The addition thereof may also be undertaken at the top of the separating column K.

The present invention further provides the diluted residue obtainable by the process of the invention.

### EXAMPLES

For examples 1 and 2, a residue R produced in the separating column K according to the example of WO 2010/066601 was used.

### Example 1

500 g of residue R was heated to 170° C. in a 1 I round-bottom flask and stirred. After 6 hours, the mixture was diluted with 500 g of ethylene glycol and cooled down while stirring.

Samples were taken during the experiment, and the viscosity was measured at 100° C.

TABLE 1
Dilution with ethylene glycol

| Time [h] | Viscosity [Pa s] |
|---|---|
| 0 | 1.12 |
| 2 | 2.43 |
| 4 | 5.22 |
| 6 | 6.86 |
| 7 | 0.04 |

### Example 2

500 g of residue R was heated to 170° C. in a 1 I round-bottom flask and stirred. After 6 hours, the mixture was diluted with 500 g of propylene glycol and cooled down while stirring.

Samples were taken during the experiment, and the viscosity was measured at 100° C.

TABLE 2
Dilution with propylene glycol

| Time [h] | Viscosity [Pa s] |
|---|---|
| 0 | 3.46 |
| 6 | 16.3 |
| 7 | 0.04 |

### Example 3

The product gas mixture from a two-stage heterogeneously catalyzed partial gas phase oxidation of (chemical grade) propylene to acrylic acid, which was conducted as described in the illustrative execution of WO 2008/090190, was subjected to a fractional condensation as in the illustrative execution of WO 2008/090190 in order to separate the acrylic acid present in the product gas mixture from the partial oxidation therefrom.

As described in the illustrative execution of WO 2008/090190, high-boiling liquid was removed from the bottom region of the condensation column and was fed to a redissociation apparatus as in the illustrative execution of WO 2010/066601.

The temperature in the bottom space of the separating column K was 168° C. The amount of residue R in the bottom space of the separating column K was about 25 m³. After 4 days, the viscosity at 120° C. rose from fluid to very viscous.

The feed of high-boiling liquid and stripping gas to the separating column K is stopped. The reflux to the separating column K is likewise stopped. Subsequently, the residue R in the bottom space of the separating column K is diluted with about 20 m³ of ethylene glycol. The diluted residue R is discharged and incinerated.

1.-15. (canceled)

16. A method of redissociating Michael adducts of acrylic acid that are present in a liquid F and have been formed in the preparation of acrylic acid, in which the liquid F comprises at least 10% by weight of Michael adducts of acrylic acid, based on the liquid F, in a redissociation apparatus comprising at least one separating column K which consists, from the bottom upward, of a bottom space, a separating space that comprises separating internals and adjoins the bottom space, and a top space adjoining the latter, and in which the pressure in the gas phase decreases from the bottom upward, an evaporator V and a pump P, in which Michael adducts present in the liquid F are split at a temperature of 130° C. to 240° C. and removed by distillation, and the remaining residue R is discharged, which comprises, in the event of an unwanted rise in the viscosity of the residue R in the bottom space of the separating column K, stopping the feed of the liquid F into the redissociation apparatus, diluting and cooling the residue R in the bottom space of the separating column K with at least 10% by volume of a solvent 1, based on the total volume of the residue R in the bottom space of the separating column K, and emptying the bottom space of the separating column K, where the solvent 1 has a boiling point at 1013 hPa of at least 150° C. and a solubility in water at 25° C. of at least 10 g per 100 g of water.

17. The method according to claim 16, wherein the liquid F comprises at least 40% by weight of Michael adducts of acrylic acid, based on the liquid F.

18. The method according to claim 16, wherein the Michael adducts present in the liquid F are split at a temperature of 155 to 180° C.

19. The method according to claim 16, wherein the solvent 1 has a boiling point at 1013 hPa of at least 210° C.

20. The method according to claim 16, wherein the solvent 1 has a solubility in water at 25° C. of at least 40 g per 100 g of water.

21. The method according to claim 16, wherein the solvent 1 is an alcohol, a carboxamide, a sulfoxide and/or a sulfone.

22. The method according to claim 16, wherein the solvent 1 is selected from ethylene glycol, propylene glycol, diethylene glycol, triethylene glycol, 2-ethoxyethanol, sulfolane, N,N-dimethylacetamide, N-methylacetamide, dimethyl sulfoxide and/or N,N-dimethylformamide.

23. The method according to claim 16, wherein the solvent 1 is a residue from the oxo process.

24. The method according to claim 16, wherein the residue R in the bottom space of the separating column K is diluted with at least 40% by volume of solvent 1, based on the total volume of the residue R in the bottom space of the separating column K.

25. The method according to claim 16, wherein the viscosity of the residue R in the bottom space of the separating column K at a temperature of 100° C. is less than 12 Pa s.

26. The method according to claim 16, wherein a stripping gas is guided into the splitting apparatus above the bottoms liquid and below the lowermost separating internals of the separating column K.

27. The method according to claim 26, wherein, in the event of an unwanted rise in the viscosity of the residue R in the bottom space of the separating column K, the feed of stripping gas into the splitting apparatus is stopped.

28. The method according to claim 16, wherein the discharged residue R is diluted with a solvent 2.

29. The method according to claim 28, wherein the solvent 2 is a residue from methanol production.

30. A diluted residue R obtained by the method of claim 16.
